(19) United States
(12) Patent Application Publication
(10) Pub. No.: US 2013/0297003 A1
(43) Pub. Date: Nov. 7, 2013

(54) Endoluminal Drug Applicator and Method of Treating Diseased Vessels of the Body

(71) Applicant: Leonard Pinchuk, Miami, FL (US)

(72) Inventor: Leonard Pinchuk, Miami, FL (US)

(73) Assignee: Innovia LLC, Miami, FL (US)

(21) Appl. No.: 13/979,403

(22) PCT Filed: Jan. 13, 2012
(86) PCT No.: PCT/US12/21299

§ 371 Date: Jul. 12, 2013

Related U.S. Application Data
(60) Provisional application No. 61/432,452, filed on Jan. 13, 2011.

(51) Int. Cl. A61F 2/95

(52) U.S. Cl. CPC A61F 2/95
USPC 623/1.12

(57) ABSTRACT

A stent-graft is coupled to an elongate flexible member at or near the distal end of the flexible member and configurable in both a collapsed configuration and an expanded configuration. The stent-graft includes an expandable stent fixed to the flexible member. A portion of the expandable stent defines a generally tubular structure in its expanded configuration. A porous polymeric mesh interfaces circumferentially about the portion of the stent defines a generally tubular structure. The mesh is expandable with the stent and carries at least one therapeutic agent. When the stent-graft is in its expanded configuration and contacts the treatment site, the at least one therapeutic agent is transferred to the treatment site by operation of contact between the stent-graft and the treatment site. The mesh can define distal and proximal openings that allow for fluid flow through the stent-graft when the stent-graft is in the expanded configuration.

### BACKGROUND OF THE INVENTION

1. Field of the Invention

The present invention relates to systems and methods for providing a treatment for diseased vessels in the body, e.g., blood vessels, aortic annulus, the bowel, etc.

2. State of the Art

Treatments for atherosclerosis have in the past included balloon angioplasty, stenting, drug-elution from a stent and recently drug delivery from a coated balloon. FIGS. 1 to 4 illustrate treatment of atherosclerosis utilizing an angioplasty balloon. FIG. 1 shows a blood vessel 1 with a proximal end 2, a distal end 3, a lumen 4 and mural thrombus or plaque 5. FIG. 2 shows a deflated angioplasty balloon 10 with a deflated balloon 11 and a proximal tip 12. FIG. 3 shows the balloon 11 inflated causing the calcification and stricture in the vessel wall to break and the thrombus or plaque 5 to be pressed against the wall of the vessel. FIG. 4 shows the blood vessel 1 with the balloon catheter removed and the plaque pressed against the wall. Note that the lumen diameter 4 is larger in FIG. 4 as compared to FIG. 1.

The problem with balloon angioplasty is that approximately 40% of vessels treated reocclude as a result of the proliferation of smooth muscle cells and subsequent narrowing of the blood vessel lumen. At first, it was hypothesized that stents would keep the vessel patent by restricting collapse of the lumen. It was found that the restenosis rate did indeed improve but it was still unreasonably high with approximately 33% occlusion by six months. It was later found that the reason for this reocclusion was due to the proliferation of smooth muscle cells in the interstices of the stent with progression to total occlusion of the lumen.

Therefore, the next attempt to inhibit restenosis involved coating the stent with an antiproliferative drug (paclitaxel or rapamycin or analogs thereof) that was released from an appropriate carrier that was coated onto the stent struts. This technology did significantly reduce the amount of restenosis to single digit rates at one year. It was then found that late stage thrombosis occurred in a small number of patients and it was hypothesized that the cause of this thrombosis was due to the thrombogenic nature of the polymeric carriers of the drug which remained on the stent when the drug was depleted or from the stent itself

It was next hypothesized that the stent may not be necessary at all if the drug can be released into the vessel wall immediately after angioplasty to prevent the smooth muscle proliferation that results in restenosis. This is especially appealing in the peripheral arteries such as the legs where stents can get inadvertently crushed if the patient, for example, crossed his/her legs Therefore researchers next turned their attention to coating balloons with drugs.

Coating a balloon with drugs raises many issues:

the balloon is normally intricately folded down onto a catheter and it is difficult to reliably coat all aspects of the balloon;

the solvents used for coating the balloon distort the balloon which could lead to poor maneuverability or premature bursting of the balloon;

the balloon is required to be inflated for long periods of time before the drug can be efficiently transferred to the vessel lumen wall, which could cause ischemia of the tissue and downstream organs which could lead to infection;

there is little room on the surface of the balloon for the amount of drug required to limit restenosis;

when the balloon is threaded through the guiding catheters and blood vessels, a large proportion of the drug may come off the balloon before it ever reaches the target; and

when the balloon is inflated, the drug flakes, cracks or otherwise does not release from the balloon in an organized predictable manner, which can lead to unpredictable results and emboli.

These issues prevent accurate dosage at the treatment site.

Devices have also been proposed for delivering an infusible drug through a fluid delivery lumen to a delivery manifold or porous construct which directs the infused drug into direct contact with the vessel wall. However, these devices also render it difficult to control the dosage of drug that is delivered to the lesion. In addition, the antiproliferative drugs commonly used for this application are not water soluble and thus would require large boluses of solvent to carry the drug and most solvents are toxic.

There is therefore a need for a better method of delivering the drug to the vessel wall that would limit restenosis.

The present application also relates to delivery drugs to a diseased heart valve. A common disease state of the heart valve occurs when the leaflets become calcified. The calcification is often times at the top of the commisures and welds the commisures together thereby restricting the complete opening of the leaflets. A procedure called valvuloplasty was developed years ago. It consists of inserting a balloon into the valve, inflating it under high pressure, and breaking apart the calcified commisures to enable them to open and close in a normal manner. This procedure is done through a small incision in the leg, with the balloon advanced though the arterial system to the heart. When successful, patients do well, and go home within a few days, avoiding the need for surgery. However, when the balloon is used, scar tissue forms and the valve re-narrows typically within 6 months, leaving the patient in the same condition as before the procedure.

The scar tissue that is formed is due to the proliferation of smooth muscle cells. The scar tissue can be minimized if an antiproliferative drug is applied to the aortic annulus at the time of inflation. This can be accomplished by coating the valvuloplasty balloon with an antiproliferative drug and releasing the drug at the time of valvuloplasty. However, many of same issues raised previously remain.

In addition, with valvuloplasty, it is possible that thrombus or plaque can dislodge from the valve area and make its way to the brain thereby causing a stroke. Similarly, during peripheral or coronary angioplasty, there is also a risk of dislodging plaque and embolizing downstream thereby causing all sorts of additional problems.

### SUMMARY OF THE INVENTION

The invention is directed to an apparatus for delivering a therapeutic agent to a treatment site of a vessel, valve, duct or bowel. The apparatus includes a first elongate flexible member having a distal end. A stent-graft is coupled to the flexible member at or near the distal end of the flexible member and configurable in both a collapsed configuration and an expanded configuration. The stent-graft includes an expandable stent fixed to the flexible member. A portion of the expandable stent defines a generally tubular structure in its expanded configuration. A porous polymeric mesh interfaces circumferentially about the portion of the stent that defines the generally tubular structure. The mesh is expandable with the stent and carries at least one therapeutic agent. When the stent-graft is in its expanded configuration and contacts the treatment site, the at least one therapeutic agent is transferred to the treatment site by operation of contact between the stent-graft and the treatment site.

In one embodiment, the mesh defines distal and proximal openings that allow for fluid flow through the stent-graft when the stent-graft is in the expanded configuration. The therapeutic agent can be selected from the group consisting of an antiproliferative drug, an antimitotic drug, and an antimigration drug.

In another embodiment, the first elongate flexible member is a guide wire.

In yet another embodiment, the first elongate flexible member is a first catheter. A second catheter defines a lumen that receives the first catheter. The first catheter is longitudinally displaceable within the lumen of the second catheter. The stent-graft is supported on a distal portion of the first catheter and extends distally beyond the distal end of the second catheter. The stent has a distal end and a proximal end. The distal end of the stent is fixed at or near the distal end of the first catheter. The proximal end of the stent is fixed to the distal end of the second catheter. The stent-graft is configured in the expanded configuration by moving the first catheter proximally relative to the second catheter, and the stent-graft is configured in the collapsed configuration by moving the first catheter distally relative to the second catheter.

In still another embodiment, the first elongate flexible member is a first catheter. A sheath covers the first catheter. The first catheter is longitudinally displaceable within the sheath. The stent-graft is supported in its collapsed configuration within a distal portion of the sheath and extends distally beyond the distal end of the first catheter. The stent has a distal end and a proximal end. The distal end of the stent is not attached to any structure. The proximal end of the stent is fixed to the distal end of the first catheter. The stent-graft is configured in the expanded configuration by moving the sheath proximally relative to the first catheter, and the stent-graft is configured in the collapsed configuration by moving the sheath distally relative to the first catheter.

In these embodiments, a balloon catheter can be longitudinally displaceable within the lumen of the first catheter. A balloon is fixed at the distal end of the balloon catheter. The balloon can have a first position in which the balloon is expanded and located distal to the stent-graft. The balloon can have a second position in which the balloon is expanded and located within the stent-graft.

In these embodiments, the apparatus can further include a second elongate flexible member having a distal end. A generally tubular porous filter element with an open distal end is deployed from the distal end of the second elongate member. The porous filter element has a collapsed configuration and an expanded configuration. At least a portion of the filter element is adapted to contact a vessel wall in its expanded configuration and block emboli from flowing into one or more vessels. The second elongate flexible member and the filter element allow for longitudinal displacement of the first elongate flexible member through the interior space of the filter element in its expanded configuration for positioning of the first elongate flexible member distally relative to the filter element.

In one embodiment, the filter element is sized to cover a branch to at least one vessel disposed distally from a contact point where it contacts the vessel wall in its expanded configuration in order to block emboli from flowing into the branch.

In another embodiment, the filter element has a self-expanding element that self-expands to a configuration where a portion of the porous filter element contacts the vessel wall.

The filter element can have a closed proximal end that captures emboli, or an open proximal end that allows emboli to escape by flowing out the open proximal end.

The filter element can be adapted to contact the wall of the ascending aorta and block emboli for reaching the arteries that feed the brain.

In another aspect, a surgical method is provided for delivering at least one therapeutic agent to a treatment site of a vessel, valve, duct or bowel, the method includes positioning the apparatus of the present application such that the stent-graft is located at the treatment site in its expanded configuration and contacts the treatment site, whereby the at least one therapeutic agent carried by the mesh is transferred to the treatment site by operation of contact between the stent-graft and the treatment site.

In one embodiment, the mesh defines distal and proximal openings that allow for fluid flow through the stent-graft when the stent-graft is in its expanded configuration. The at least one therapeutic agent can be selected from the group consisting of an antiproliferative drug, an antimitotic drug, and an antimigration drug.

In another embodiment, a balloon can be expanded within the stent-graft in its expanded configuration while the stent-graft is contacting the treatment site. This can aid in transferring the therapeutic agent(s) carried by the mesh to the treatment site.

In yet another aspect, a surgical method for delivering at least one therapeutic agent to a treatment site of a vessel, valve, duct or bowel, is provided that employs a stent-graft configurable in both a collapsed configuration and an expanded configuration. The stent-graft includes an expandable stent, wherein a portion of the expandable stent defines a generally tubular structure in the expanded configuration. A porous polymeric mesh interfaces circumferentially about the portion of the stent that defines the tubular structure and is expandable with the stent. At least one therapeutic agent carried by the mesh. The stent-graft is located at the treatment site in its expanded configuration such that it contacts the treatment site, whereby the at least one therapeutic agent is transferred to the treatment site by operation of contact between the stent-graft and the treatment site. The mesh defines distal and proximal openings that allow for fluid flow through the stent-graft when the stent-graft is in its expanded configuration. The therapeutic agent can be selected from the group consisting of an antiproliferative drug, an antimitotic drug, and an antimigration drug. A balloon can be within the stent-graft in its expanded configuration while the stent-graft is contacting the treatment site in order to aid in the transfer of the therapeutic agent(s) carried by the mesh to the treatment site.

### BRIEF DESCRIPTION OF THE DRAWINGS

FIG. 1 is a schematic diagram of a diseased vessel with restenosis.

FIGS. 2 to 3 are schematic illustration of a balloon catheter performing balloon angioplasty according to the prior art.

FIG. 4 is a schematic diagram of the diseased vessel of FIG. 1 after the balloon angioplasty of FIGS. 2 to 3.

FIGS. 5 to 7 illustrate a first embodiment of a drug delivery apparatus according to the present application.

FIGS. 8 to 9 illustrate a second embodiment of a drug delivery apparatus according to the present application.

FIGS. 10 to 12 illustrate an embodiment of a balloon catheter that is used in conjunction with the apparatus of FIGS. 8 and 9.

FIGS. 13 and 14 illustrate alternate embodiments of a drug delivery apparatus according to the present application.

FIG. 15 is a schematic illustration of the human heart.

FIG. 16 is a simplified schematic view of the aorta and left ventricle of the heart.

FIGS. 17 to 22 illustrate an embodiment of a deployment catheter and emboli filter element that is deployed within the aortic arch and used in conjunction with the apparatus of FIGS. 8 to 12 to apply at least therapeutic agent to a diseased aortic valve and protect against emboli entering the arteries that feed the brain.

FIG. 23 illustrates an alternate embodiment of the apparatus of FIGS. 8 to 9 for use in conjunction with the deployment catheter and emboli filtering element of FIGS. 17 to 22.

FIG. 24 illustrates an alternate embodiment of the emboli filtering element of FIGS. 17 to 22.

FIG. 25 illustrates an apparatus that is deployed within the aortic arch and used to apply at least therapeutic agent to a diseased aortic valve and protect against emboli entering the arteries that feed the brain.

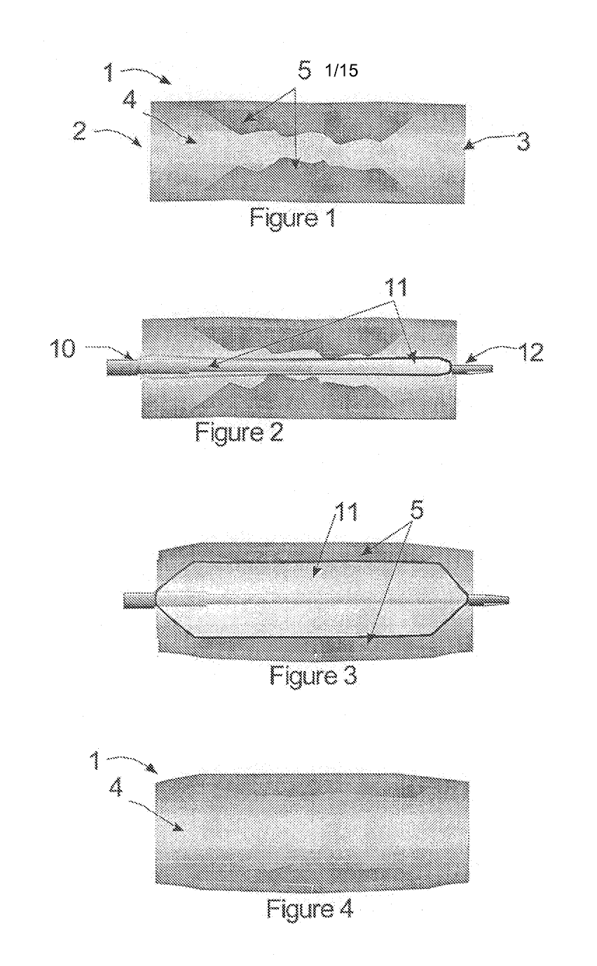

### DETAILED DESCRIPTION OF THE PREFERRED EMBODIMENTS

As used herein, the term “distal” is generally defined as in the direction of the heart of the patient, or away from a user of the system/apparatus/device. Conversely, “proximal” generally means in the direction away from the heart of the patient, or toward the user of the system/apparatus/device.

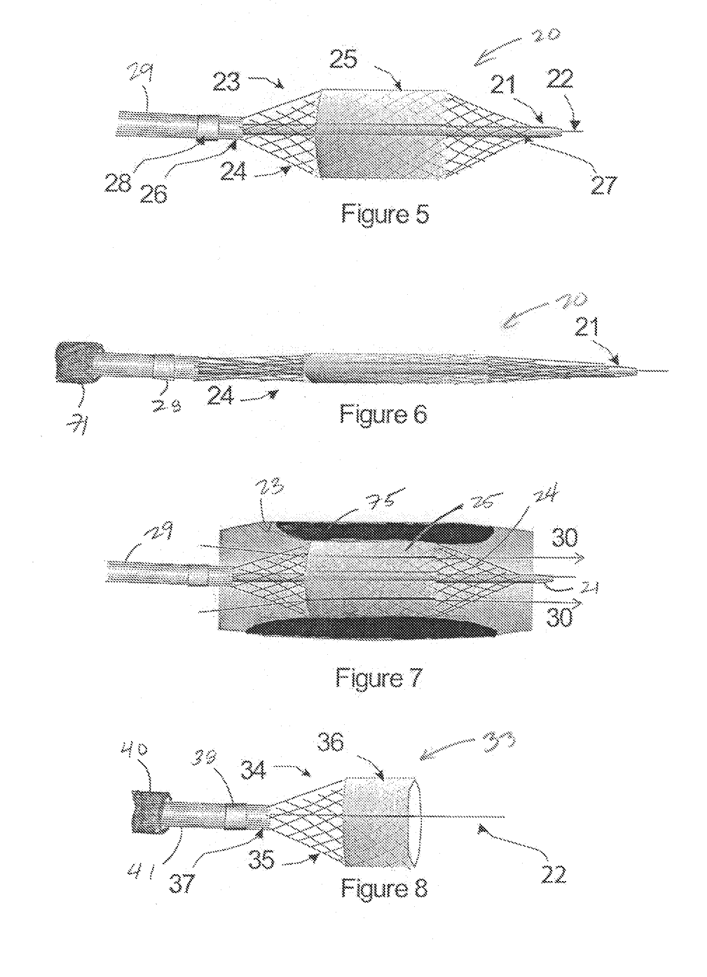

Turning now to FIGS. 5 and 6, there is shown one embodiment of a drug delivery apparatus 20 according to the present application. The apparatus 20 includes a first catheter 21 that defines a central lumen which can receive and follow a guide wire 22. A second catheter 29 defines a central lumen that receives the first catheter 21 and allows the first catheter 21 to move inside the central lumen distally and proximally relative to the second catheter 29. The first catheter 21 and the second catheter 29 are both flexible in nature such that they can be maneuvered through the tortious pathway of the vasculature during use. A stent-graft-like construction 23 (referred to herein as stent-graft 23) is supported on the distal portion of the first catheter 21 which extends beyond the distal end of the second catheter 29. The stent-graft 23 includes a polymeric mesh 25 that is fixed to (or integrally formed on) an expandable stent 24. The stent 24 includes a network of filaments with interstitial spaces therebetween. The distal end of the stent 24 is fixed to the first catheter 21 at location 27 (which is at or near the distal end of the first catheter 21). The proximal end of the stent 24 is fixed to the second catheter at location 26 (which is at or near the distal end of the second catheter 29). The stent 24 can be fixed to the catheters 29 and 21 by first placing a mandrill inside each catheter, then placing the stent over the catheter in the area to be attached, then placing a temporary heat shrink Teflon tube over the stent and then fusing the stent to the catheter by heating the Teflon tube in a hot clamshell mold to the melting point of the catheter material. Forces from the heat shrink Teflon as well as forces from the clamshell cause the filaments of the stent to push into the melted catheter material. The assembly is then cooled and the Teflon tube removed. The stent is thereby fixed to the catheter in this manner. Other suitable fixation methods can also be used.

The stent 24 is expandable from a collapsed (i.e. low-profile) configuration (FIG. 6) to an expanded configuration (FIG. 5) by proximal movement of the first catheter 21 relative to the second catheter 29. It is also collapsible from the expanded configuration (FIG. 5) to the collapsed configuration (FIG. 6) by distal movement of the first catheter 21 relative to the second catheter 29. The mesh 25 expands and collapses with the stent 24.

The mesh 25 can interface to the inner surface of the stent 24, while leaving exposed the outer surface of the stent 24. The mesh 25 can also interface to the outer surface of the stent 24, while leaving exposed the inner surface of the stent 24. The mesh 25 can also interface to the both the outer surface and inner surface of the stent 24 and thus cover portions of both the outer surface and inner surface of the stent 24. A radio-opaque marker 28 can be placed at or near the distal end of the second catheter 29 for positioning using fluoroscopy. Similarly, a radio-opaque marker (not shown) can be placed at or near the distal end of the first catheter 21 for positioning using fluoroscopy. One or more radio-opaque markers (not shown) can also be placed in or on the stent 24 to help positioning using fluoroscopy.

The expanded configuration of the stent 24 can define a generally tubular structure (such as a central cylindrical portion) with frustoconical end portions as shown in FIG. 5. The mesh 25 can interface to the generally tubular structure of the stent 24, while leaving open at least part of the frustoconical end portions of the stent 24 as shown in FIG. 5. In this arrangement, the distal and proximal ends of the mesh 25 define respective distal and proximal openings. Blood can flow into and through the stent-graft 23 by entering through the open filaments of the distal frustoconical end portion of the stent, through the distal opening of the mesh 25, out the proximal opening of the mesh 25, and out the open filaments of the proximal frustoconical end portion of the stent 24 as represented by the arrows 30 in FIG. 7. The collapsed configuration of the stent 24 of FIG. 6 preferably provides a maximal cross-sectional diameter through the stent-graft 23 that is less than or equal to the outer diameter of the second catheter 29.

The mesh 25 is comprised of a porous polymeric material suitable for carrying a therapeutic agent, such as a porous electrostatically spun polyurethane. The mesh 25 is preferably 0.1 mm to 0.001 mm in thickness, and more preferably 0.01 mm in thickness. The therapeutic agent can be vacuum impregnated into the porous structure of mesh 25, either neat or in a carrier (such as gelatin, albumin, polysaccharide, carbohydrate, dextran, polymers, hydrogels, surface modifying agents, for example fluorine or silicone containing polyolefins or other suitable carrier). Alternatively, the therapeutic agent can be mixed in with the solution of material that will be spun into the mesh, and spun with the mesh as it is formed. The dried mesh thus formed will thereby be loaded with the therapeutic agent wherein the agent will elute from the mesh when the mesh is contacted with the vessel to be treated. The therapeutic agent is preferably not water or blood soluble, and is preferably transferable to tissue via a lipophilic property. The porous structure of the mesh 25 can allow for blood to pass through the mesh 25. A membrane (not shown) can line the inner surface of the stent 24 or mesh 25, where the membrane functions to prevent passage blood through the mesh 25. The membrane can also function to prevent migration of the therapeutic agent to blood flowing within the blood vessel and through the stent 24 or mesh 25.

The mesh 25 can carry one or more therapeutic agents such as an antiproliferative drug, an antimitotic drug, and an antimigration drug. Examples of such therapeutic agents include mitomycin C, 5-fluorouracil, corticosteroids (corticosteroid triamcinolone acetonide is most common), modified toxins, methotrexate, adriamycin, radionuclides (e.g., such as disclosed in U.S. Pat. No. 4,897,255, herein incorporated by reference in its entirety), protein kinase inhibitors (including staurosporin, which is a protein kinase C inhibitor, as well as a diindoloalkaloids and stimulators of the production or activation of TGF-beta, including tamoxifen and derivatives of functional equivalents, e.g., plasmin, heparin, compounds capable of reducing the level or inactivating the lipoprotein Lp(a) or the glycoprotein apolipoprotein(a) thereof), nitric oxide releasing compounds (e.g., nitroglycerin) or analogs or functional equivalents thereof, paclitaxel or analogs or functional equivalents thereof (e.g., taxotere or an agent based on Taxol®, whose active ingredient is paclitaxel), inhibitors of specific enzymes (such as the nuclear enzyme DNA topoisomerase II and DAN polymerase, RNA polyermase, adenl guanyl cyclase), superoxide dismutase inhibitors, terminal deoxynucleotidyl-transferas, reverse transcriptase, antisense oligonucleotides that suppress cell proliferation, angiogenesis inhibitors (e.g., endostatin, angiostatin and squalamine), rapamycin, everolimus, zotarolimus, cerivastatin, and flavopiridol and suramin and the like.

Other examples of therapeutic agents include the following: peptidic or mimetic inhibitors, such as antagonists, agonists, or competitive or non-competitive inhibitors of cellular factors that may trigger proliferation of cells or pericytes (e.g., cytokines (for example, interleukins such as IL-1), growth factors (for example, PDGF, TGF-alpha or -beta, tumor necrosis factor, smooth muscle- and endothelioal-derived growth factors such as endothelin or FGF), homing receptors (for example, for platelets or leukocytes), and extracellular matrix receptors (for example, integrins).

Representative examples of useful therapeutic agents in the category of agents that address cell proliferation include: subfragments of heparin, triazolopyrimidine (for example, trapidil, which is a PDGF antagonist), lovastatin; and prostaglandins E1 or I2.

Several of the above and numerous additional therapeutic agents appropriate for the practice of the present invention are disclosed in U.S. Pat. Nos. 5,733,925 and 6,545,097, both of which are herein incorporated by reference in their entirety.

A shown in FIG. 6, a guide catheter or sheath 71 can be provided to position the apparatus 20 within the vasculature. The guide catheter 71 defines a central lumen that receives the second catheter 29 (as well as the first catheter 21 and the guide wire 22) and allows the second catheter 29 (as well as the first catheter 21 and the guide wire 22) to move inside the central lumen distally and proximally relative to the guide catheter 71. The guide catheter 71 is flexible in nature such that it can be maneuvered through the tortious pathway of the vasculature during use. The stent 24 can be more lubricious than the mesh 25. Thus, locating the mesh 25 on the inner surface of the stent 24 with the outer surface of the stent 24 exposed can allow the outer surface of the stent 24 to function as a bearing to facilitate displacement of the stent-graft 23 as it is advanced through the guide catheter 71. Furthermore, locating the mesh 25 along the inside of the stent 24 minimizes opportunities for the therapeutic agent carried by the mesh 25 to be inadvertently removed by contact with the guide catheter 71. Still further, having the stent 24 on the outside (with mesh 25 on the inside) allows the stent 24 to dent into or score the vessel wall which will help in the penetration of the therapeutic agent(s) carried by the mesh 25 transfer into the vessel wall as well as cut some tissue that may be causing stricture of the vessel and relieve the stricture.

During use, the guide wire 22 is introduced into the vasculature and maneuvered through the vasculature to a position at or near the treatment site (e.g., the site of an atherosclerotic lesion). The guide catheter 71 is introduced into and maneuvered through the vasculature over the guide wire 22 to a position at or near the treatment site. The apparatus 20 (first catheter 21 and second catheter 29) with the stent-graft 23 in its collapsed configuration (FIG. 6) is introduced into and maneuvered through the vasculature over the guide wire 22 and through the guide catheter 71 to a position at or near the treatment site. In the collapsed configuration of FIG. 6, the first catheter 21 is offset from the distal end of the second catheter 29 such that the stent 24 elongates and simultaneously reduces the maximal cross-sectional diameter of the stent-graft 23. In the preferred embodiment, the maximal cross-sectional diameter of the stent-graft 23 in the collapsed configuration is less than the outer diameter of second catheter 29 in order to facilitate maneuvering the apparatus 20 into place. With the stent-graft 23 located at or near the treatment site, the stent-graft 23 is expanded into its expanded configuration (FIG. 5) by proximal movement of the first catheter 21 relative to the second catheter 29 such that the stent-graft 23 contacts the vessel wall at the treatment site and the therapeutic agent carried by the mesh 25 of the stent-graft 23 is transferred to the treatment site for therapeutic purposes.

FIG. 7 shows the stent-graft 23 in place in a blood vessel where the stent-graft 23 contacts the vessel wall at the treatment site 75 (and the mesh 25 is positioned adjacent to the treatment site 75). Note that when fully deployed, blood is free to travel through the open frustoconical ends of stent 25 (particularly through the interstices of the frustoconical ends of the stent 25 as shown by arrows 30) and thereby perfuse the distal extremities and not cause ischemia.

The stent-graft 23 is advantageous in respect to balloons in that the porous polymeric structure of the mesh 25 can be filled with a large quantity of therapeutic agent(s), the mesh 25 will prevent the therapeutic agent(s) that it carries from pealing or flaking off in the guiding catheter 71, and the mesh 25 deforms uniformly in a predictable manner. In addition, the open nature of the stent 24, at its proximal and distal ends, allows the mesh 25 to be deployed for a long period of time without causing ischemia as blood can pass through the open ends of the stent 24 and perfuse the distal circulatory system when the stent is expanded into its expanded configuration into contact against the vessel wall. Once the therapeutic agent(s) is eluted from the mesh 25, the stent-graft 23 can be removed from the vasculature in the reverse order to which it was introduced. In addition, the filaments of the stent 24 may be structured to score the vessel wall. This allows the drug to penetrate deeper into the tissue of the vessel wall.

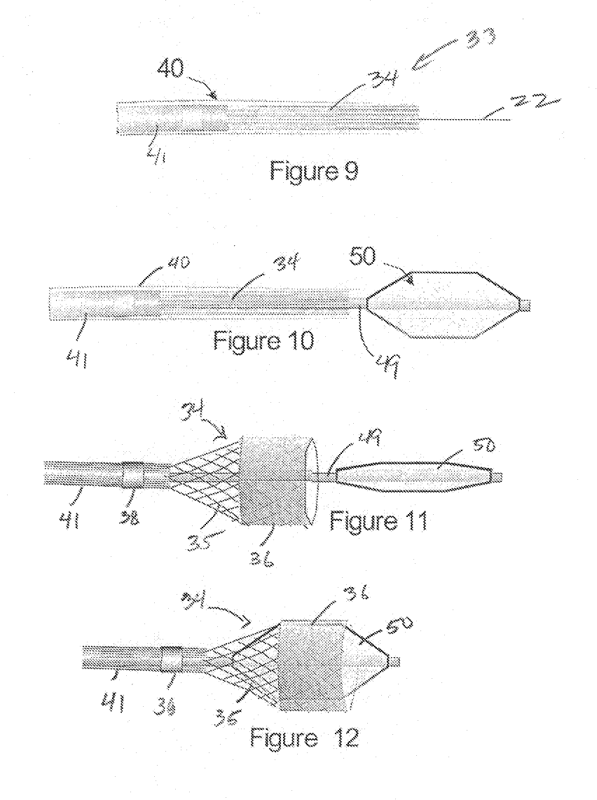

FIGS. 8 and 9 show another embodiment of a drug delivery apparatus according to the present application. The apparatus 33 includes a catheter 41 that defines a central lumen which can receive and follow a guide wire 22. The catheter 41 is flexible in nature such that they can be maneuvered through the tortious pathway of the vasculature during use. A stent-graft-like construction 34 (referred to herein as “stent graft 34) is supported by the distal end of the catheter 41 and extends beyond the distal end of the catheter 41. The stent-graft 34 includes a polymeric mesh 36 that is fixed to (or integrally formed on) an expandable stent 35. The stent 35 includes a network of filaments with interstitial spaces therebetween. The proximal end of the stent 35 is fixed to the catheter 41 at location 37 (which is at or near the distal end of the catheter 41). The stent 35 can be fixed to the catheter 41 by first placing a mandrill inside the catheter, then placing the stent over the catheter in the area to be attached, then placing a temporary heat shrink Teflon tube over the stent and then fusing the stent to the catheter by heating the Teflon tube in a hot clamshell mold to the melting point of the catheter material. Forces from the heat shrink Teflon as well as forces from the clamshell cause the filaments of the stent to push into the melted catheter material. The assembly is then cooled and the Teflon tube removed. The stent 35 is thereby fixed to the catheter 41 in this manner. Other suitable fixation mechanisms can also be used. The distal end of the stent-graft 34 is open and not attached to any structure. As shown in FIG. 9, an outer sheath 40 defines a central lumen whose distal portion receives the stent-graft 34 (as well as the guide wire 22). The outer sheath 40 is flexible in nature such that it can be maneuvered through the tortious pathway of the vasculature during use.

With the stent-graft 34 disposed inside the distal portion of the lumen of the outer sheath 40, the stent 35 has a collapsed (i.e. low-profile) configuration as shown in FIG. 9. The stent-graft 34 is deployed from the distal portion of the lumen of the outer sheath 40 by moving the outer sheath 40 proximally relative to the catheter 41. In this deployed position, the stent 35 can expand to an expanded configuration as shown in FIG. 8. The stent 35 can be self-expandable (or possibly expanded by a balloon or other suitable expansion mechanism). It is also collapsible from the expanded configuration (FIG. 8) to the collapsed configuration (FIG. 9) by returning the stent-graft 34 back into the distal portion of the lumen of the outer sheath 40 by moving the outer sheath 40 distally relative to the catheter 41. The mesh 36 expands and collapses with the stent 35.

The mesh 36 can interface to the inner surface of the stent 35, while leaving exposed the outer surface of the stent 35. The mesh 36 can also interface to the outer surface of the stent 35, while leaving exposed the inner surface of the stent 35. The mesh 36 can also interface to the both the outer surface and inner surface of the stent 35 and thus cover portions of both the outer surface and inner surface of the stent 35. A radio-opaque marker 38 can be placed at or near the distal end of the catheter 41 for positioning using fluoroscopy. One or more radio-opaque markers (not shown) can also be placed in or on the stent 35 to help positioning using fluoroscopy.

The expanded configuration of the stent 35 can define generally tubular structure (i.e., a cylindrical portion) with a proximal frustoconical end portion as shown in FIG. 8. The mesh 36 can interface to the cylindrical portion of the stent 35, while leaving open at least part of the proximal frustoconical end portion of the stent 35 as shown in FIG. 9. In this arrangement, the distal and proximal ends of the mesh 36 define respective distal and proximal openings. Blood can flow into and through the stent-graft 34 by entering through the distal opening of the mesh 36, out the proximal opening of the mesh 36, and out the open filaments of the proximal frustoconical end portion of the stent 35. The collapsed configuration of the stent 35 provides a maximal cross-sectional diameter through the stent-graft 34 that is less than the diameter of the distal portion of the lumen of the outer sheath 40.

The mesh 36 is comprised of a porous polymeric material suitable for carrying a therapeutic agent, such as a porous electrostatically spun polyurethane. The mesh 36 is preferably 0.1 mm to 0.001 mm thick, and more preferably 0.01 mm thick. The therapeutic agent can be vacuum impregnated into the porous structure of mesh 36, either neat or in a carrier (such as gelatin, albumin, polysaccharide, carbohydrate, dextran, polymers, hydrogels, surface modifying agents, for example fluorine or silicone containing polyolefins or other suitable carrier). Alternatively, the therapeutic agent can be mixed in with the solution of material that will be spun into the mesh, and spun with the mesh as it is formed. The dried mesh thus formed will thereby be loaded with the therapeutic agent wherein the agent will elute from the mesh when the mesh is contacted with the vessel to be treated. The therapeutic agent is preferably not water or blood soluble, and is preferably transferable to tissue via a lipophilic property. The porous structure of the mesh 36 can allow for blood to pass through the mesh 36. A membrane (not shown) can line the inner surface of the stent 35 or mesh 36, where the membrane functions to prevent passage blood through the mesh 36. The membrane can also function to prevent migration of the therapeutic agent to blood flowing within the blood vessel and through the stent 35 or mesh 36.

The mesh 36 can carry one or more therapeutic agents as described above for the mesh 25.

The stent 35 can be more lubricious than the mesh 36. Thus, locating the mesh 36 on the inner surface of the stent 35 with the outer surface of the stent 35 exposed can allow the outer surface of the stent 35 to function as a bearing to facilitate displacement of the stent-graft 34 as it is deployed from the distal portion of the lumen of the outer sheath 40. Furthermore, locating the mesh 36 along the inside of the stent 35 minimizes opportunities for the therapeutic agent carried by the mesh 36 to be inadvertently removed by contact with the distal portion of the outer sheath 40.

During use, the guide wire 22 is introduced into the vasculature and maneuvered through the vasculature to a position at or near the treatment site (e.g., the site of an atherosclerotic lesion). With the stent-graft 34 housed within the distal portion of the lumen of the outer sheath (FIG. 9), the outer sheath 40 and catheter 41 are introduced into and maneuvered through the vasculature over the guide wire 22 to a position at or near the treatment site. The stent-graft 34 is deployed from the distal portion of the lumen of the outer sheath 40 by moving the outer sheath 40 proximally relative to the catheter 41. With the stent-graft 34 located at or near the treatment site, the stent-graft 34 expands into its expanded configuration (FIG. 8) such that the stent-graft 34 contacts the vessel wall at the treatment site and the therapeutic agent carried by the mesh 36 of the stent-graft 34 is transferred to the treatment site for therapeutic purposes.

The lumen of the catheter 41 of FIGS. 8 and 9 can receive a balloon catheter 49 that supports an expandable balloon 50 at its distal end as shown in FIGS. 10, 11 and 12. With the stent-graft 34 disposed within the lumen of the outer sheath 40, the balloon 50 can be placed distally relative to the outer sheath 40 as shown in FIG. 10. In this configuration, the balloon 50 can be expanded to dilate the treatment site to facilitate passing the outer sheath 40 to within the dilated treatment site. Once the outer sheath 40 is advanced to the treatment site, the balloon 50 can be positioned distally from the outer sheath 40 and the stent-graft 34 can be deployed from the distal portion of the lumen of the outer sheath 40 by moving the outer sheath 40 proximally relative to the catheter 41. This configuration (without the vessel) is shown in FIG. 11. With the stent-graft 34 deployed from the outer sheath 40 and located at the treatment site, the stent-graft 34 expands into its expanded configuration (FIG. 8) such that the stent-graft 34 contacts the vessel wall at the treatment site and the therapeutic agent carried by the mesh 36 of the stent-graft 34 is transferred to the treatment site for therapeutic purposes.

The balloon 50 can be positioned inside of the stent-graft 34 (with the stent-graft 34 in its deployed and expanded configuration) as shown in FIG. 12. The balloon 50 can be expanded such that the stent-graft 34 dilates with the balloon 50 and presses against the vessel wall at the treatment site. Such dilation can aid in transferring the therapeutic agent from the mesh 36 of the stent-graft 34 to the treatment site. It is appreciated that the balloon 50 may thereafter be collapsed and drawn back into the catheter 41 or displaced distally of the stent-graft (to the relative position shown in FIG. 11) to permit blood flow through the stent-graft 34 while the stent-graft 34 remains expanded in contact against the vessel wall tissue for an additional period of time.

It can also be appreciated that the stent-graft 34 can be manufactured and heat set so that its natural position is in its collapsed state wherein the sheath 40 on catheter 41 is not required. The deflated balloon 50 can be positioned within collapsed stent-graft 34, the assembly located in the lesion to be treated and both the balloon 50 and stent-graft 34 expanded together to both dilate the vessel as well as transfer the therapeutic agent simultaneously. It is also contemplated that the balloon catheter 49/50 can be used in a similar manner with the apparatus 20 of FIGS. 5 to 7.

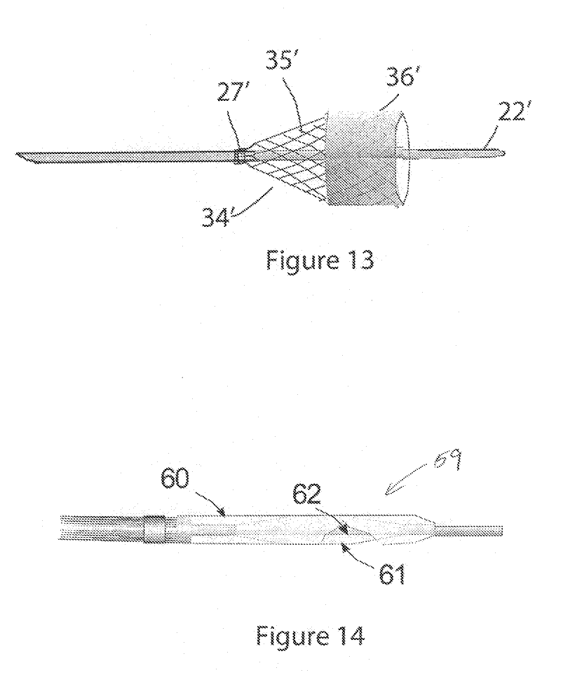

FIG. 13 shows a further embodiment of the invention, where the stent-graft 34′ (stent 35′ and mesh 36′) is integrally attached to a guidewire 22′ at site 27′; there is no catheter on which the stent is mounted. The distal end of stent 35′ supports the mesh 36′. The stent may be used sequentially with an expansion device which first performs angioplasty. The expansion device may be a balloon catheter with a lumen that permits the guide wire 22′ with stent 25′ to be passed therethrough. After the angioplasty, the expansion device can be withdrawn (for example, back into a delivery catheter), and the stent 25′ is expanded into its expanded configuration as shown in FIG. 13. In this configuration, the stent-graft 34′ contacts the vessel wall at the treatment site and the therapeutic agent carried by the mesh 36′ of the stent-graft 34′ is transferred to the treatment site for therapeutic purposes. Alternatively, where the expansion device has no lumen for receiving the guide wire 22′ with stent 25′, the expansion device may first be withdrawn from the patient and then the guide wire 22′ with stent 25′ can be advanced to the treatment location. The stent 25′ can be constructed of a shape memory material so as to permit self-expansion when delivered to the treatment site. Such self-expansion can be as a result of an elastic or superelastic quality or by stimulation to an expanded memory form upon application of energy such as heat.

FIG. 14 is still another embodiment of a drug delivery apparatus according to the present application. The apparatus 59 does not employ a stent. The apparatus 59 includes a tubular mesh 60 that is secured around a deflated balloon 61. The mesh 60 is comprised of a porous polymeric material suitable for carrying a therapeutic agent, such as a porous electrostatically spun polyurethane. The therapeutic agent can be vacuum impregnated into the porous structure of mesh 60, either neat or in a carrier (such as gelatin, albumin, polysaccharide, carbohydrate, dextran, polymers, hydrogels, surface modifying agents, for example fluorine or silicone containing polyolefins or other suitable carrier). Alternatively, the therapeutic agent can be mixed in with the solution of material that will be spun into the mesh, and spun with the mesh as it is formed. The dried mesh thus formed will thereby be loaded with the therapeutic agent wherein the agent will elute from the mesh when the mesh is contacted with the vessel to be treated. The therapeutic agent is preferably not water or blood soluble, and is preferably transferable to tissue via a lipophilic property. The mesh 60 can carry one or more therapeutic agents as described above for the mesh 25. When the balloon 61 is inflated, the porous mesh 60 dilates with it and releases the therapeutic agent(s) that it carries. Cutout 62 shows the balloon 61 under the mesh 60. The mesh 60 can be attached to the catheter or attached directly to the balloon 61. The mesh 60 is removed from the vasculature with the balloon 61 once the therapeutic agent has been deployed.

The stents described in this application can be made from metal; either self expanding or balloon expanding. Exemplary metals are Nitinol, Elgiloy, MP35N, superalloy, titanium and the like. Exemplary balloon-expandable stents include stainless steel, gold, platinum, tantalum and the like. The stent can also be made from polymers such as PET, Nylon, PEEK, PEEKEK, polyimine, polyurethane, polyethylene, polypropylene, fluropolymers and the like as long as it has sufficient memory to self-expand when released from the sheath.

In another aspect of the present application, the drug delivery apparatus of the present application can be used to apply one or more therapeutic agents to a diseased heart valve.

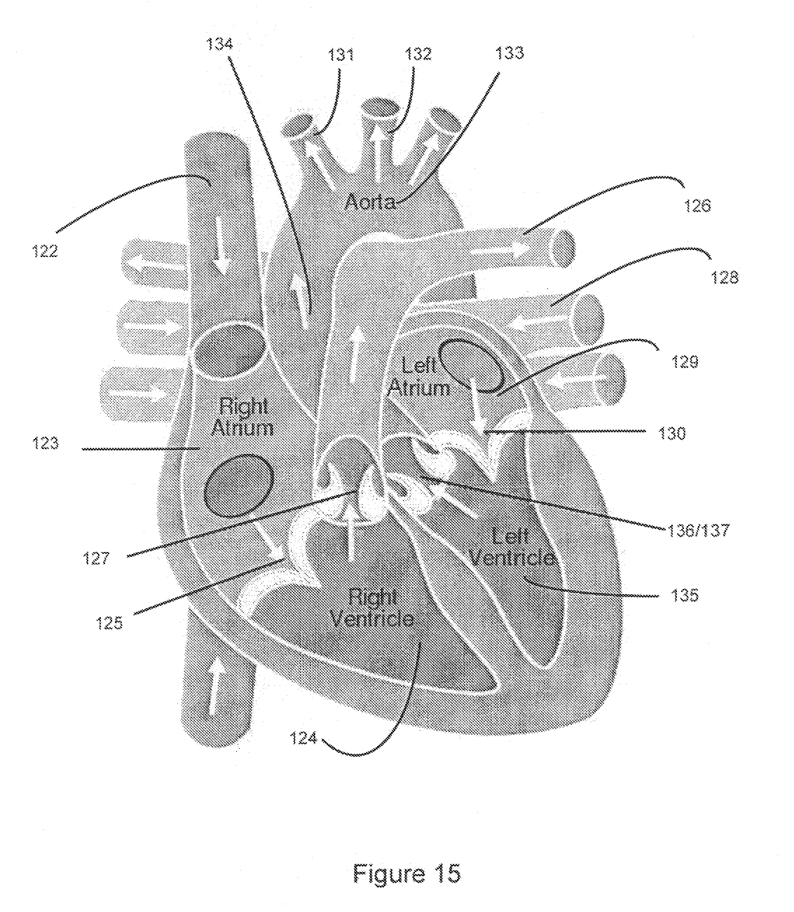

Turning to FIG. 15, the human heart has four chambers, two superior atria (the right atrium 123 and the left atrium 129) and two inferior ventricles (the right ventricle 124 and the left ventricle 135). The atria (the right atrium 123 and the left atrium 129) are the receiving chambers and the ventricles (the right ventricle 124 and the left ventricle 135) are the discharging chambers. The pathway of blood through the human heart consists of a pulmonary circuit and a systemic circuit. Deoxygenated blood is supplied from the body and flows through the superior vena cava 122 into the right atrium 123 and is pumped into the right ventricle 124 through the tricuspid valve 125. The deoxygenated blood in the right ventricle 124 is pumped to the pulmonary arteries 126 through the pulmonary valve 127 for supply to the lungs. The lungs oxygenate the blood. The oxygenated blood flows from the lungs through the pulmonary veins 128 into the left atrium 129, where it is pumped into the left ventricle 135 through the mitral valve 130. The oxygenated blood is pumped from the left ventricle 135 through the aortic valve 136, 137 into the aorta for supply to the body. The aorta distributes oxygenated blood to all parts of the body through the systemic circulation.

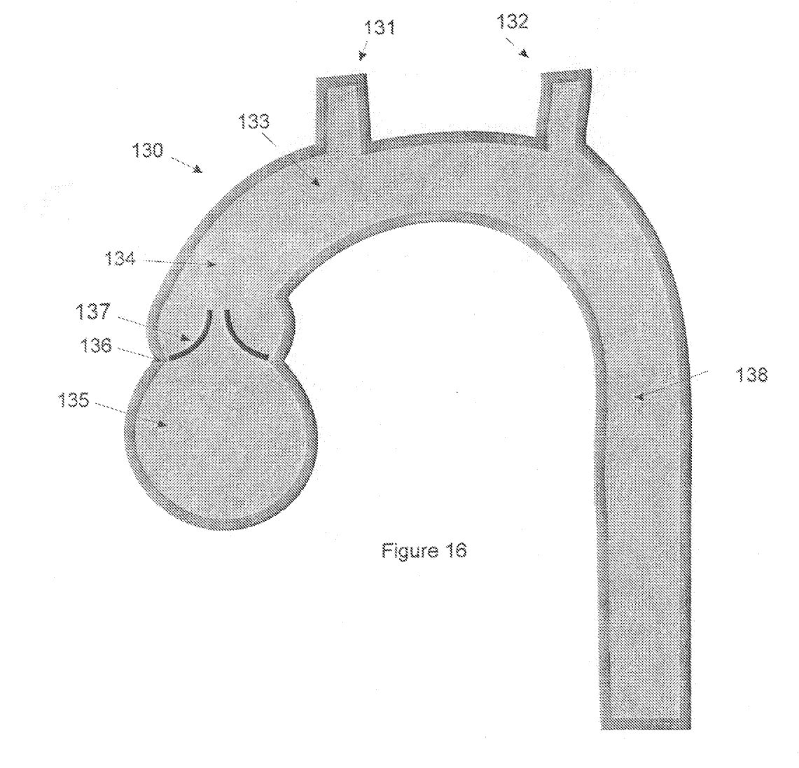

The aorta can be logically divided into three segments/sections including the ascending aorta, the aortic arch and the descending aorta. The ascending aorta (labeled 134 in FIG. 15) extends between the aortic valve 136/137 and the aortic arch (labeled 133 in FIG. 15). The aortic arch 133 is shaped like an inverted U and includes branches to arteries that supply oxygenated blood to the brain. Specifically, the brachiacephalic artery 131, the left common carotid artery 132 and the left subclavian artery branch off from the aortic arch 133. The left subclavian artery is not labeled in FIG. 15—it is the artery that branches off the aortic arch 33 next to the left common carotid artery 132 and may at times be fused to the left common carotid and thereby appear as one artery leaving the aortic arch 133. These three arteries will herein be collectively called the “arteries feeding the brain.” The ascending aorta 134 is filled with oxygenated blood by contraction of the left ventricle 135 which pushes blood past aortic valve 136/137. The aortic valve includes an annulus 136 from which is attached aortic valve leaflets 137. Oxygenated blood travels up the ascending aorta 134, through the aortic arch 133 and down the descending aorta (labeled 138 in FIG. 16) to the kidneys and lower parts of the body. FIG. 16 is a simplified schematic illustration of the aorta as well as the aortic valve and left ventricle of the heart.

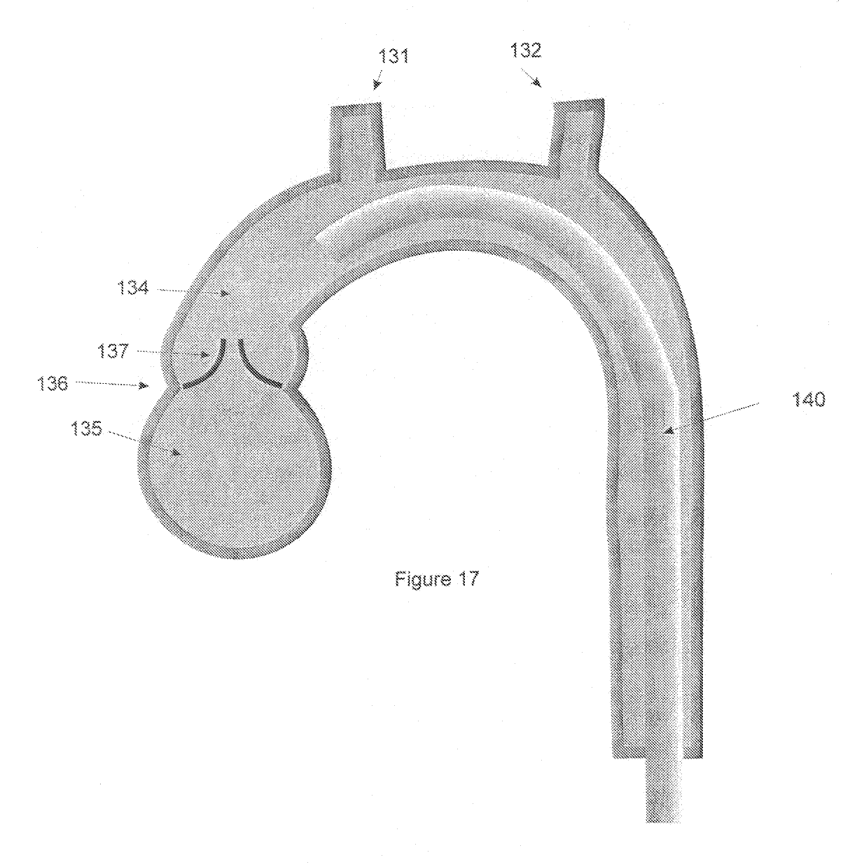

FIGS. 17 to 22 show another embodiment of a drug delivery apparatus according to the present application. The apparatus is used to delivery one or more therapeutic agents to a diseased aortic valve of the heart. The apparatus includes a delivery catheter 140 that defines a central lumen which can receive and follow a guide wire (not shown). The delivery catheter 140 is flexible in nature such that it can be maneuvered through the tortious pathway of the vasculature during use. A filter element 150 is supported by the distal end of a support tube 145 within the lumen of the delivery catheter 140. The support tube 145 extends proximally within the delivery catheter 140 and is flexible in nature such that it can be maneuvered through the tortious pathway of the vasculature during use. The filter element 150 is a tubular porous mesh structure similar in construction to the stent-grafts described previously but with a porosity sufficiently large to allow blood to pass through it, while blocking the flow of particulate matter such as emboli that can cause a stroke in the event that it travels into the arteries feeding the brain and lodges in the brain. The filter element 150 can be comprised of a porous polymeric material, such as a porous electrostatically spun polyurethane. The effective pore size of the mesh should be in the range of 1 to 10 microns to prevent larger emboli from reaching the brain.

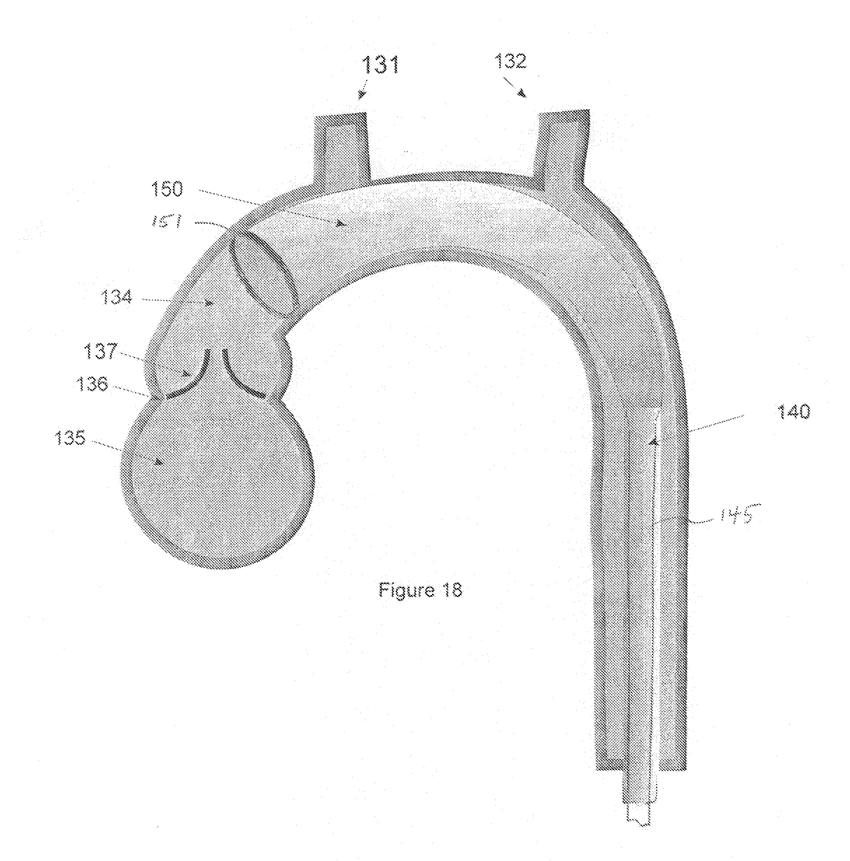

A distal portion of the filter element 150 (such as the distal rim) can include a self-expandable structure 151 that self-expands to an expanded configuration in contact the wall of the ascending aorta 134 as shown in FIGS. 18 to 22. The self-expandable structure 151 can be realized from one or more self-expanding elastic materials, such as Nitinol, Elgiloy, MP35N, superalloy, titanium and the like. It can also made from polymers such as PET, Nylon, PEEK, PEEKEK, polyimine, polyurethane, polypropylene, polyethylene and the like as long as it has sufficient memory to self-expand when deployed. Alternatively, the filter element 150 can be self-expanding metal or polymeric braid with a spun-coat mesh covering the braid to reduce its pore size.

The filter element 150 is loaded into the distal portion of the lumen of the delivery catheter 140 with the self-expandable structure 141 in a collapsed configuration. The filter element 150 is deployed from the distal portion of the lumen of the delivery catheter 140 by moving the delivery catheter 140 proximally relative to the support tube 145 of the filter element 150. Other suitable deployment mechanisms can also be used. In the deployed configuration, the self-expandable member 151 of the filter member 150 self-expands to an expanded configuration as shown in FIGS. 18 to 22. The member 151 is also collapsible from the expanded configuration to the collapsed configuration by returning the filter element 150 back into the distal portion of the lumen of the delivery catheter 150 by moving the delivery catheter 140 distally relative to the support tube 145.

The tubular filter element 150 is sized such that when it is placed into contact with the wall of the ascending aorta 134, the filter element 150 extends distally past at least the arteries feeding the brain (1231, 132) and protects the arteries feeding the brain from receiving emboli released from upstream. More specifically, with the distal rim 151 of the filter element 150 contacting the wall of the ascending aorta 134, the filter element 150 prohibits any embolus caused by dislodgement of a thrombus or plaque at the aortic valve treatment site from passing around the seal and into the protected branch(es) of the vasculature—i.e., the arteries feeding the brain.

The delivery catheter 140 and filter element 150 supported therein are used in conjunction with the drug delivery apparatus of FIGS. 8 and 9 to delivery one or more therapeutic agent(s) to the aortic valve.

More specifically, the filter element 150 is loaded into the distal portion of the lumen of the delivery catheter 140 with the self-expandable structure 151 in a collapsed configuration, and the deployment catheter 140 is introduced into and maneuvered through the vasculature (possibly over a guide wire not shown) such that its distal portion is positioned in the ascending aorta 134 as shown in FIG. 17. The filter element 150 is deployed from the distal portion of the lumen of the delivery catheter 140 by moving the delivery catheter 140 proximally relative to the support tube of the filter element 150. Other suitable deployment mechanisms can also be used. In the deployed configuration, the self-expandable member 151 self-expands to an expanded configuration and contacts the will of the aorta as shown in FIG. 18.

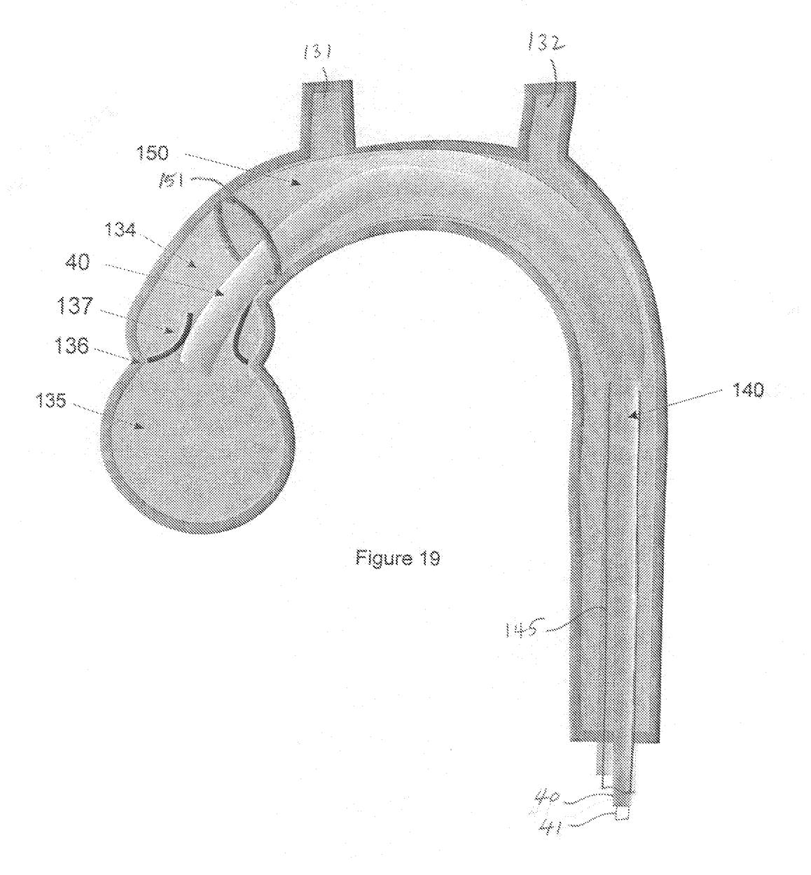

With the stent-graft 34 housed within the distal portion of the lumen of the outer sheath (FIG. 9), the outer sheath 40 and the catheter 41 are introduced into and maneuvered through the delivery catheter 140 (and the support tube 145 therein) and possibly over a guide wire (not shown) such that the distal end of the outer sheath 40 is position at or near the treatment site (e.g., at or near the annulus 136 and contacting the valve leaflets 137 as shown in FIG. 19).

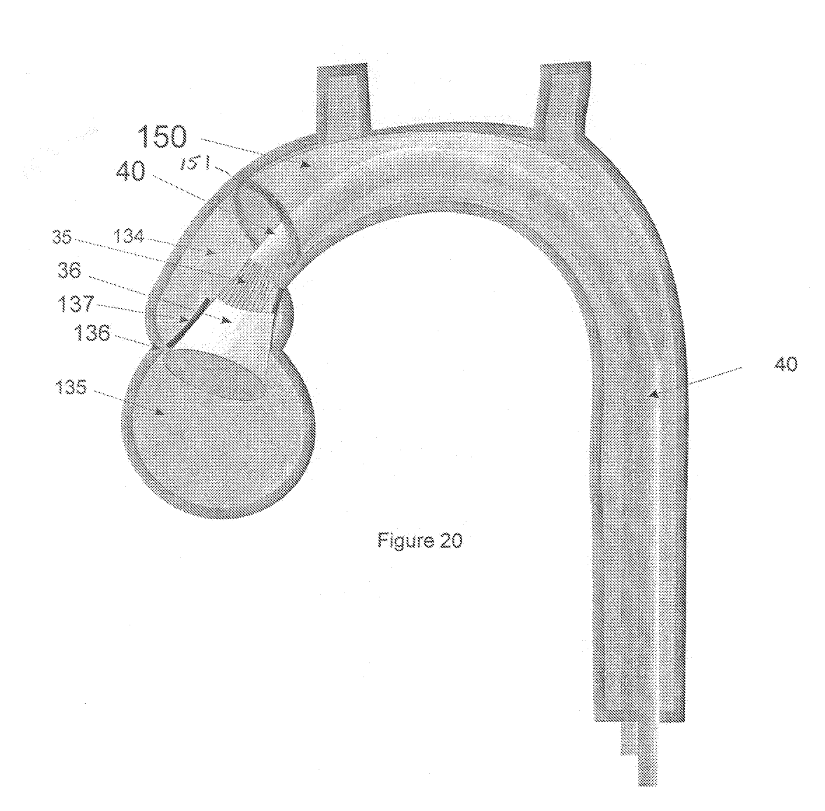

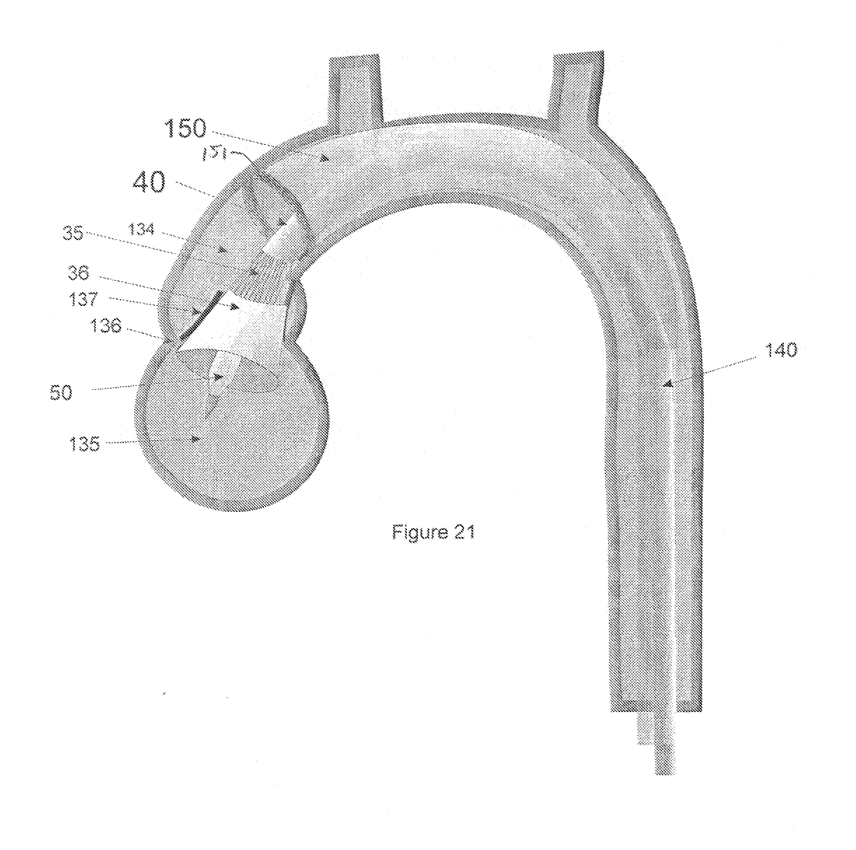

The stent-graft 34 is deployed from the distal portion of the lumen of the outer sheath 40 by moving the outer sheath 40 proximally relative to the catheter 41. With the stent-graft 34 located at or near the treatment site, the stent-graft 34 expands into its expanded configuration such that the stent-graft 34 contacts the vessel wall at the treatment site as shown in FIG. 20 and the therapeutic agent carried by the mesh 36 of the stent-graft 34 is transferred to the treatment site for therapeutic purposes.

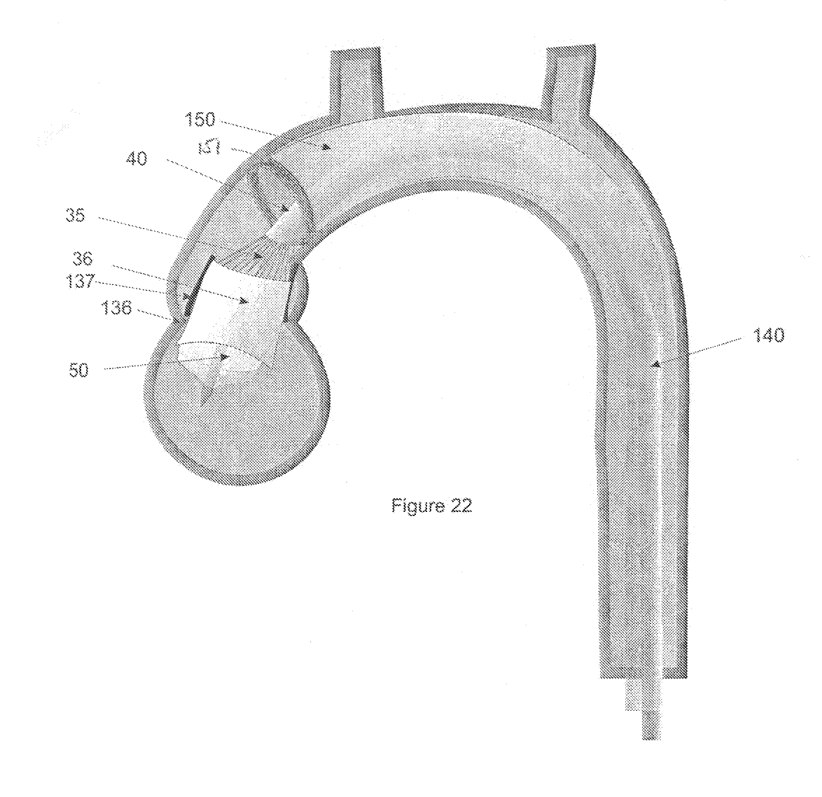

The lumen of the catheter 41 can receive a balloon catheter 49 that supports an expandable balloon 50 at its distal end as shown in FIGS. 10, 11 and 12. The balloon 50 can be positioned inside of the stent-graft 34 (with the stent-graft 34 in its deployed and expanded configuration) as shown in FIG. 21. The balloon 50 can be expanded such that the stent-graft 34 dilates with the balloon 50 and presses against the valve leaflets 137 and the annulus 136 of the aortic valve as shown in FIG. 22. Such dilation can aid in transferring the therapeutic agent from the mesh 36 of the stent-graft 34 to the treatment site as well as simultaneously performing a valvuloplasty procedure wherein the calcified leaflets fusing the leaflets together at the commisures are detached from each other. It is appreciated that the balloon 50 may thereafter be collapsed and drawn back into the catheter 41 or displaced distally of the stent-graft while the stent-graft 34 remains expanded in contact against the vessel wall tissue for an additional period of time to further allow transfer of therapeutic agent.

One skilled in the art will realize that debris or volatile plaque (emboli) can be dislodged from the procedure performed on the annulus 36 or the valve leaflets 37. The filter element 150 captures the emboli and protects them from flowing into the arteries feeding the brain. In this manner, the emboli will flow into the filter element 50 and be diverted away from entering the arteries feeding the brain, thereby preventing an inadvertent stroke. The emboli captured by the filter element 150 can be aspirated out of delivery catheter 140 or it can reside in the filter element 150 and removed when delivery catheter 140 and the filter element 150 are removed from the body at the end of the procedure.

It will also be appreciated that the drug delivery apparatus described above with respect to FIGS. 5 to 7 can also be used in a similar manner in conjunction with the delivery catheter 140 and filter element 150 to apply one or more therapeutic agent(s) to the diseased aortic valve.

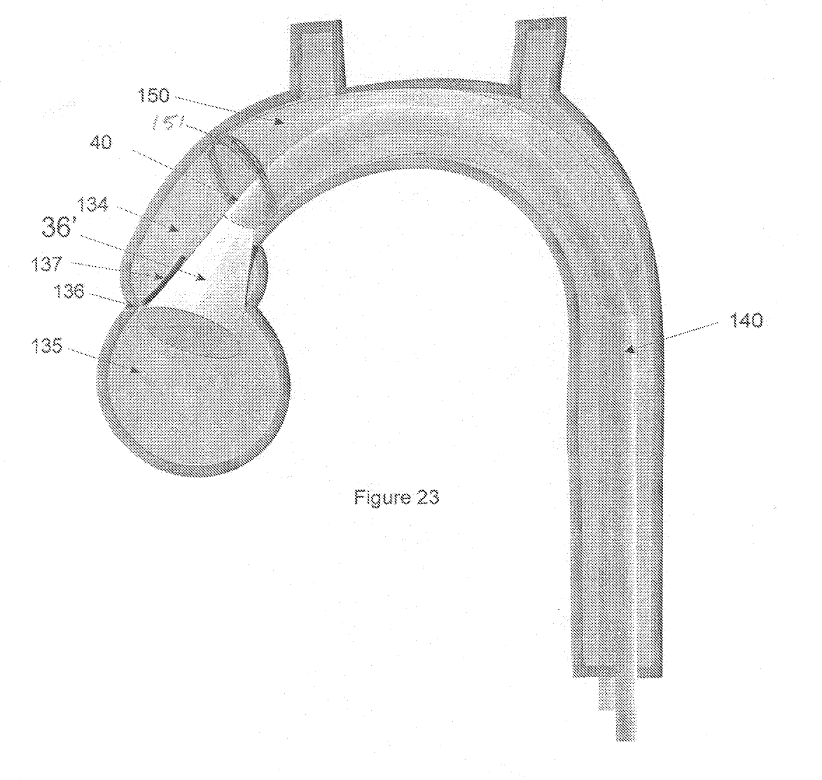

FIG. 23 illustrates an alternate embodiment of the stent-graft of the present application. In this embodiment, the mesh 36′ of the stent-graft 34 covers the proximal frustoconical end of the stent 35. This embodiment reduces blood flow through the lumen of the stent-graft 34 in the deployed configuration of the stent-graft 34.

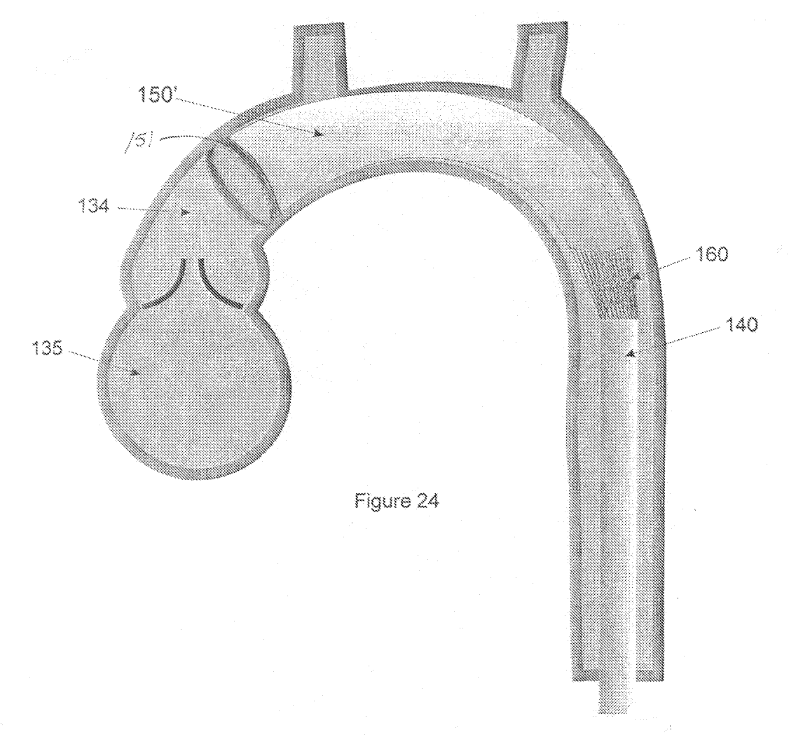

FIG. 24 illustrates an alternate embodiment of the catheter 140 which incorporates a mesh 160 of larger porosity at its proximal end to enable more blood flow during the procedure. Blood will flow through the lumen of filter element 150 and out through open mesh 160. In this embodiment, some emboli may flow distally past the mesh 160 were it can be managed in some other manner (for example, by allowing it to break down in transit through the vasculature or possibly lodge in other parts of the vasculature where damage to the patient will be less severe than if the emboli were to travel to the brain).

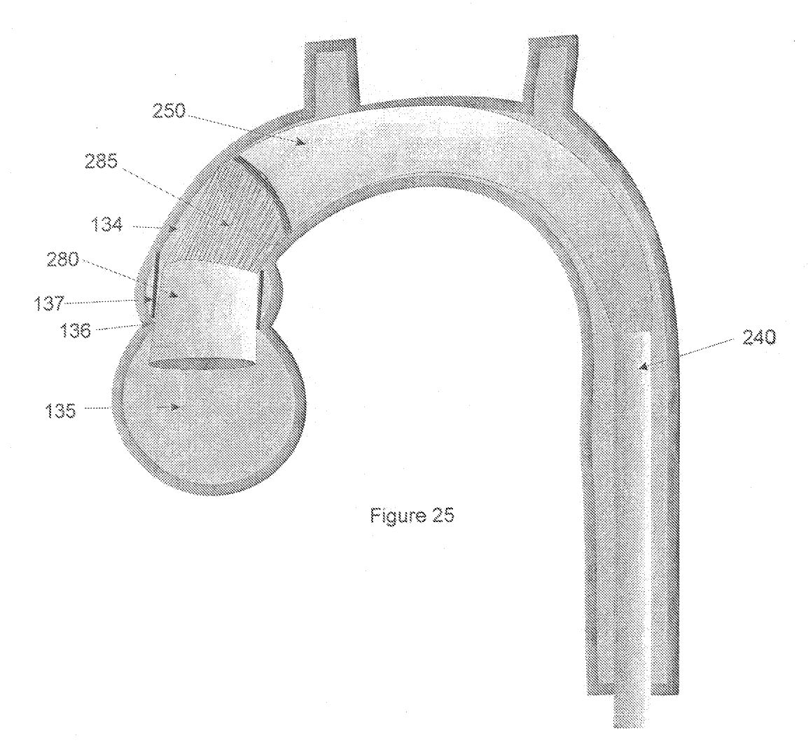

FIG. 25 illustrates an alternate embodiment of a drug delivery apparatus according to the present invention. In this embodiment, a stent-graft 285/280 (which is equivalent to the stent-graft 34 described herein) is fixed to the distal end of a filter element 250 (which is analogous to the filter element 150 described herein). Both of these elements are supported within the distal portion of a lumen of a delivery catheter 240 (which is equivalent to the catheter 140 described herein). In this embodiment, the stent-graft 285/280 and filter element 250 are deployed one after the other from the delivery catheter 240 at the treatment site adjacent the diseased aortic valve. Emboli dislodged from the vicinity of the annulus 36 and leaflets 37 can flow through open structure 285 and enter filter element 250 and be diverted from the arteries feeding the brain. A balloon can be fed through the catheter 140 and filter element 150 to the inside of stent-graft 280 and inflated to release the drug carried in the mesh of the stent-graft 280 such that it transfers to the annulus 136 and the leaflets 137 of the aortic valve. The catheter described in FIG. 25 is made using one continuous stent where the porosity of the mesh differs along the length of the stent. The pore size of filter area 250 may be 5 to 20 microns in diameter to enable blood flow to the brain, yet deflect emboli. The porosity of the therapeutic agent delivery mesh 280 may be smaller to provide a higher density of material (0.1 to 10 microns) to trap and deliver the therapeutic agent.

The catheters and like tubular members described herein can employ proximal handles that allow for manipulation of the position of the catheter and tubular members relative to one another as well as a proximal inflation port that provides for supply of pressured fluid for inflation of a balloon (if an inflatable balloon is used).

There have been described and illustrated herein several embodiments of an apparatus and method for delivering an endoluminal drug applicator to a treatment site, using the applicator at the treatment site as well as removing the apparatus from the vasculature. While particular embodiments of the invention have been described, it is not intended that the invention be limited thereto, as it is intended that the invention be as broad in scope as the art will allow and that the specification be read likewise. For example, the systems and methods of the present application described above for applying therapeutic agent(s) to an aortic valve can be used to apply therapeutic agent(s) to other valves of the heart (such as the tricuspid valve 125 and the pulmonary valve 127 where the system is positioned in the superior vena cava 122 and the mesh containing the therapeutic agent is positioned within either valve. In this embodiment the filter is not used as the lungs are natural traps for emboli and filtering is not necessary. The system can also be used in the mitral valve 130 where the catheter is entered into the pulmonary vein 128 and the mesh containing the therapeutic agent is positioned in the mitral valve. In this procedure, the filter element is placed in the aortic arch via another catheter that is maneuvered from the femoral artery in the groin. The systems and methods of the present application described above for capturing (or diverting) emboli can be also be used for any stenotic artery to prevent volatile plaque from embolizing downstream.

The systems and methods of the present application described above can also be used in the bowel to deliver chemo agents or actinic radiation to treat cancers of the colon. Similarly, it can be used to treat infection or other diseases of the bowel such as irritable bowel syndrome or Crones disease. Similarly, these aforementioned catheter systems can be used to treat bronchial, bile ducts, lachrymal ducts, etc. where local delivery of a therapeutic agent can be beneficial. It will therefore be appreciated by those skilled in the art that yet other modifications could be made to the provided invention without deviating from its spirit and scope as claimed.

1. An apparatus for delivering a therapeutic agent to a treatment site of a vessel, valve, duct or bowel, the apparatus comprising:
a) a first elongate flexible member having a distal end; and
b) a stent-graft coupled to said flexible member at or near the distal end of said flexible member and configurable from a collapsed configuration to an expanded configuration, said stent-graft including,
i) an expandable stent fixed to said flexible member, wherein a portion of said expandable stent defines a generally tubular structure in said expanded configuration,
ii) a porous polymeric mesh that interfaces circumferentially about said portion of said stent and is expandable with said stent, and
iii) at least one therapeutic agent carried by said mesh, wherein when said stent-graft is in said expanded configuration and contacts the treatment site, said at least one therapeutic agent is transferred to the treatment site by operation of contact between said stent-graft and the treatment site.

2. An apparatus according to claim 1, wherein:
said mesh defines distal and proximal openings that allow for fluid flow through said stent-graft when said stent-graft is in said expanded configuration.

3. An apparatus according to claim 1, wherein:
said at least one therapeutic agent is selected from the group consisting of an antiproliferative drug, an antimitotic drug, and an antimigration drug.

4. An apparatus according to claim 1, wherein:
said first elongate flexible member is a guidewire.

5. An apparatus according to claim 1, wherein:
said first elongate flexible member is a first catheter.

6. An apparatus according to claim 5, further comprising:
a second catheter that defines a lumen that receives said first catheter, said first catheter longitudinally displaceable within the lumen of said second catheter, wherein said stent-graft is supported on a distal portion of said first catheter that extends distally beyond the distal end of said second catheter.

7. An apparatus according to claim 6, wherein:
said stent has a distal end and a proximal end, the distal end of said stent fixed at or near the distal end of said first catheter, and the proximal end of said stent fixed to the distal end of said second catheter.

8. An apparatus according to claim 7, wherein:
said stent-graft is configured in said expanded configuration by moving said first catheter proximally relative to said second catheter, and said stent-graft is configured in said collapsed configuration by moving said first catheter distally relative to said second catheter.

9. An apparatus according to claim 5, further comprising:
a sheath that covers that said first catheter, said first catheter longitudinally displaceable within said sheath, wherein said stent-graft is supported in its collapsed configuration within a distal portion of said sheath and extends distally beyond the distal end of said first catheter.

10. An apparatus according to claim 9, wherein:
said stent has a distal end and a proximal end, the distal end of said stent not attached to any structure, and the proximal end of said stent fixed to the distal end of said first catheter.

11. An apparatus according to claim 10, wherein:
said stent-graft is configured in said expanded configuration by moving said sheath proximally relative to said first catheter, and said stent-graft is configured in said collapsed configuration by moving said sheath distally relative to said first catheter.

12. An apparatus according to claim 5, further comprising:
a balloon catheter longitudinally displaceable within the lumen of said first catheter, said balloon catheter having a distal end; and
a balloon fixed at said distal end of said balloon catheter.

13. An apparatus according to claim 12, wherein:
said balloon has a first position in which said balloon is expanded and located distal said stent-graft.

14. An apparatus according to claim 13, wherein:
said balloon has a second position in which said balloon is expanded and located within said stent-graft.

15. An apparatus according to claim 1, further comprising:
c) a second elongate flexible member having a distal end; and
d) a generally tubular porous filter element with an open distal end that is deployed from the distal end of said second elongate member, said porous filter element having a collapsed configuration and an expanded configuration, wherein at least a portion of said filter element is adapted to contact a vessel wall in its expanded configuration and block emboli from flowing into one or more vessels.

16. An apparatus according to claim 15, wherein:
said second elongate flexible member and said filter element allow for longitudinal displacement of the first elongate flexible member through the interior space of said filter element in its expanded configuration for positioning of said first elongate flexible member distally relative to said filter element.

17. An apparatus according to claim 15, wherein:
said filter element is sized to cover a branch to at least one vessel disposed proximally from a contact point where said filter element contacts the vessel wall in its expanded configuration in order to block emboli from flowing into said branch.

18. An apparatus according to claim 15, wherein:
said filter element has a self-expanding element that self-expands to a configuration where a portion of the porous filter element contacts the vessel wall.

19. An apparatus according to claim 15, wherein:
said filter element has a closed proximal end that captures emboli.

20. An apparatus according to claim 15, wherein:
said filter element has an open proximal end that allows emboli to escape by flowing out the open proximal end.

21. An apparatus according to claim 15, wherein:
said filter element is adapted to contact the wall of the ascending aorta and block emboli from reaching the arteries that feed the brain.

22. A surgical method for delivering at least one therapeutic agent to a treatment site of a vessel, valve, duct or bowel, the method comprising:
a) providing the apparatus of claim 1; and
b) positioning the apparatus of claim 1 such that said stent-graft is located at the treatment site in said expanded configuration and contacts the treatment site, whereby said at least one therapeutic agent is transferred to the treatment site by operation of contact between said stent-graft and the treatment site.

23. A surgical method according to claim 22, wherein:
said mesh defines distal and proximal openings that allow for fluid flow through said stent-graft when said stent-graft is in said expanded configuration.

24. A surgical method according to claim 22, wherein:
said at least one therapeutic agent is selected from the group consisting of an antiproliferative drug, an antimitotic drug, and an antimigration drug.

25. A surgical method according to claim 1, further comprising:
c) expanding a balloon within said stent-graft in its expanded configuration while said stent-graft is contacting the treatment site.

26. A surgical method for delivering at least one therapeutic agent to a treatment site of a vessel, valve, duct or bowel, the method comprising:
a) providing a stent-graft configurable in both a collapsed configuration and an expanded configuration, said stent-graft including,
i) an expandable stent, wherein a portion of said expandable stent defines a generally tubular structure in said expanded configuration,
ii) a porous polymeric mesh that interfaces circumferentially about said portion of said stent and expandable with said stent, and
iii) at least one therapeutic agent carried by said mesh; and
b) locating said stent-graft at the treatment site in said expanded configuration such that it contacts the treatment site, whereby said at least one therapeutic agent is transferred to the treatment site by operation of contact between said stent-graft and the treatment site, wherein said mesh defines distal and proximal openings that allow for fluid flow through said stent-graft when said stent-graft is in said expanded configuration.

27. A surgical method according to claim 26, wherein:
said at least one therapeutic agent is selected from the group consisting of an antiproliferative drug, an antimitotic drug, and an antimigration drug.

28. A surgical method according to claim 26, further comprising:
c) expanding a balloon within said stent-graft in its expanded configuration while said stent-graft is contacting the treatment site.
